(19) United States
(12) Patent Application Publication
(10) Pub. No.: US 2007/0196913 A1
(43) Pub. Date: Aug. 23, 2007

(54) Detachable bottom of cell culture plates for slides

(76) Inventor: Stephen Liye Chen, El Monte, CA (US)

Correspondence Address: Stephen Liye Chen, 12331 Felipe Street, El Monte, CA, 91732, US

(21) Appl. No.: 11/357,592

(22) Filed: Feb. 18, 2006

(51) Int. Cl. C12M 1/22

(52) U.S. Cl. 435/305.1; 435/288.3

(57) ABSTRACT

An improvement made to cell culture plates and dishes. The plastic bottom is made detachable for slides without using glass for cell culture. To detach a plate bottom, the user inserts a key to the plate and makes a 90-degree turn, which breaks a detaching ring and releases the plastic bottom as a circular disc with attached cells on it.

### FIELD OF THE INVENTION

The present invention relates generally to cell culture containers. More specifically, it relates to a device and method for saving cultured cells to microscope slides.

### BACKGROUND OF THE INVENTION

Cell culture has become one of the most important means in modern biotechnology. It can be classified into attached and suspended types of cultures. In attached cultures, pieces of tissues or dispersed cells attach to the bottom of a culture container under a layer of liquid culture medium, which enables researchers to perform their experiments.

The physical property of the bottom is one of the most important parameters effecting the behavior of attached cells in culture. Many cell types just do not grow well on certain types of bottoms, such as a glass bottom. Therefore, high quality culture containers are made with plastics, such as polystyrene.

Cell culture is a two-step process, a healthy culture, and a way to save the culture as permanent slide for microscope analysis. Tyndorf et al, in U.S. Pat. No. 5,882,922, teaches a plate for generating good cultures. But, Tyndorf et al has failed to demonstrate how to make a slide from the plate. MatTek Corporation, from its website, www.glass-bottom-dishes.com, teaches culture dishes with glass bottom. Unfortunately, MatTek sacrifices culture quality for making slides. Stevens et al, in U.S. Pat. No. 5,605,813, also teaches a culture bottom using microscope slide, which is usually made in glass. Stevens et al has failed to make his culture slide bigger for more applications. Stevens et al has further failed to simplify his culture slide. Glue is introduced to his culture, which is a positive addition to cultured cells.

A device and method for both cell culture and slide making are highly desired but remains unsolved.

### SUMMARY OF THE PRESENT INVENTION

It is, therefore, an object of the invention to provide a device and method for both cell culture and slide making.

The advance of the invention over prior arts can be summarized as follows:
(1) A detachable plastic bottom ensures a healthy cell culture. The invention uses polystyrene as cell culture container.
(2) The plastic bottom makes slides directly. The plastic bottom is detachable easily and is used to form slides.

### BRIEF DESCRIPTION OF THE DRAWINGS

FIG. 1 is a sectional side view of a traditional multi-well culture plate.

FIG. 2a and 2b illustrate a preferred improvement of the multi-well culture plate.

FIG. 3a and 3b show how to form a slide using the detached bottom.

FIG. 4a and 4b show applications of the improvement to different dishes.

### DETAILED DESCRIPTION OF PREFERRED EMBODIMENT

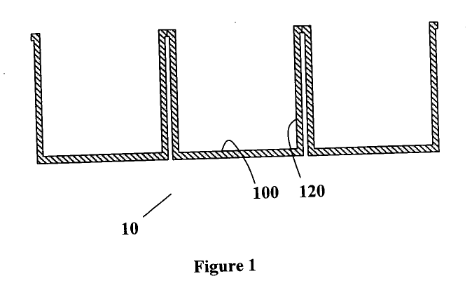

FIG. 1 shows a sectional side view of a traditional plate 10 having three culture wells in a row. Multi-well plates are popular for their space-saving and easy handling features. Bottom 100 provides a flat surface for cell attachment. Wall 120, rising around bottom 100, holds liquid culture medium in each well. Traditional plates, however, are not designed for bottom detachment to make slides.

Detachment of plate bottom is a challenge. First, polystyrene bottom is strong and is not easy to break by hand. And the second, no disturbance of attached cells is acceptable.

The preferred improvement of present invention has addressed the challenge by teaching a detaching ring to reduced strength and a levering mechanism for force-enhancement.

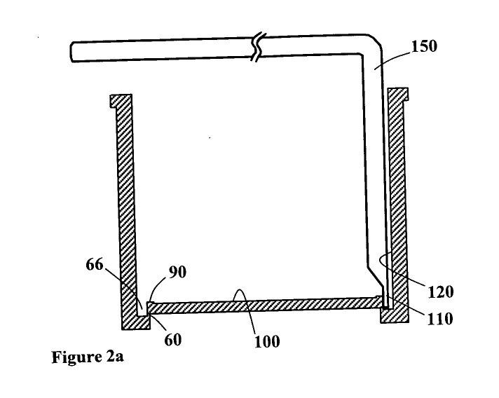

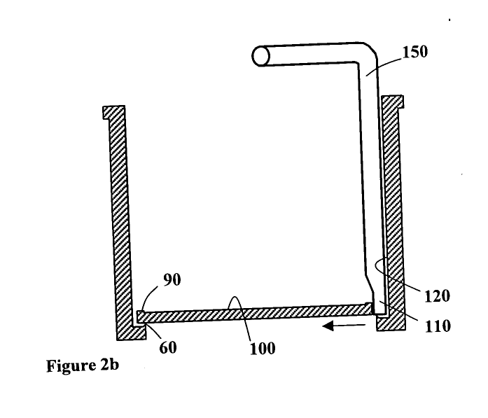

FIG. 2a illustrates a preferred improvement showing how to make bottom 100 easily detachable. A notch 66, in a dimension of 3 mm deep and 3 mm wide, is distributed around edge of bottom 100, A region of notch 66 under bottom 100 is reduced to 0.3 mm in thickness, which is named as detaching ring 60. Detaching ring 60 controls a desired breaking line by a self-destruction under an external force. For generating a nice edge of bottom 100 after detachment, the vertical height of detaching ring 60 should be less than 0.5 mm.

It should be noticed that detaching ring 60 is still too sturdy for forced destruction if being pushed vertically from top or bottom. But it will be otherwise easily destructible if it is pushed horizontally from left or right utilizing a levering mechanism.

To make a detachment, the basic steps are:
1. Insert a “I” shape key head 110 into notch 66 between bottom 100 and wall 120, as shown in FIG. 2a.
2. Turn key 150 horizontally, which generates a slow, smooth, but extremely powerful horizontal force against bottom 100, as shown by arrow in FIG. 2b.

Here wall 120 is used as a supporting point in the levering mechanism. The movement of key 150 breaks detaching ring 60 and pushes detached bottom 100 off the center, as shown in FIG. 2b. Another advantage can be seen from FIG. 2b, that is, detached bottom 100 with attached cells remains inside the well, which prevents cells from accidental damages. With the improvement and a simple hand-held tool, users can make detachment quickly and easily.

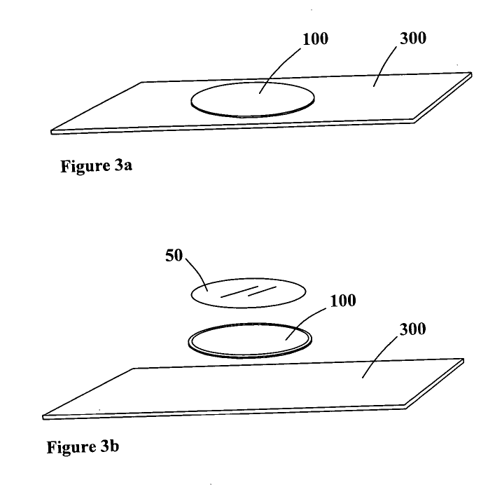

FIG. 3a and 3b show how to make a slide. Detached bottom 100 looks like a circular disc with cells attached to one side. It can be used as a coverslip and mounted directly onto a glass slide 300, as shown in FIG. 3a. The side with attached cells is placed down against slide 300. Alternatively, a very thin circular cover glass 50 can be used on top of detached bottom 100 to cover cells. Then detached bottom 100 can be placed on glass slide 300, as shown from FIG. 3b illustrative diagram.

Multi-well plates and dishes have a variety of diameters. But slides are fixed in one format. That is, some modifications should be made to adapt the invention in different embodiments.

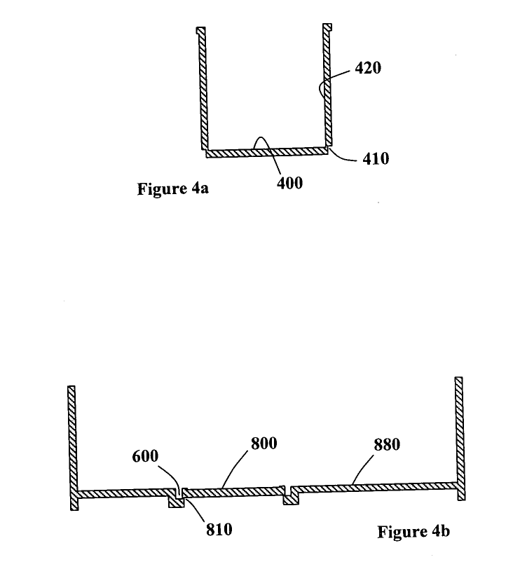

FIG. 4a shows a detaching ring 410 being built between a bottom 400 and a wall 420 for multi-well plates. Gaps between neighboring wells can be used to support a lever for force enhancement. Alternatively, a cutting wheel can be used to break detaching ring 410. FIG. 4b shows only a small portion of a bottom 880 being detachable for making slide. A detaching ring 810 defines a detachable region 800 in a diameter of 22 mm to fit slides. A notch 600 permits an insertion of a lever head to detach region 800 horizontally.

Although the descriptions above contains specifications, it is apparent to those who skilled in the art that a number of other variations and modifications can be made to the invention without departing from its spirit and scope. Notch 66, for example, can be omitted, detaching ring 60 can be built in a any shape, such as a hexagon. A hexagon wrench then can be used for its detachment. Detaching ring 60 can also be built on lower portion of wall 120 instead of bottom 100, which can be detached by a cutting wheel. Therefore, the descriptions as set out above should not be constructed as limiting the scope of the invention but as merely providing illustration of the presently preferred embodiment of the invention.

1. An improvement to a container having a bottom for cell attachment in a culture, comprising:
A detaching member, incorporated with said container, defining at least a portion of said bottom, having a reduced thickness to control a line of breakage, being destructible by an external force.

2. An improvement to a container having a bottom for cell attachment in a culture, comprising:
A detaching member, as a part of said container using a same raw material, defining at least a portion of said bottom, having a reduced thickness to control a line of breakage, being destructible by an external force for detachment of said portion of said bottom from said container.

3. A method for detaching at least a portion of a bottom from a container after a cell culture, comprising steps of:
(a) having a tool and an improvement to said container, said improvement comprising;
a detaching member, as a part of said container using a same raw material, defining at least a portion of said bottom, having a reduced thickness to control a line of breakage, being destructible by an external force for detachment of said portion of said bottom from said container;
(b) engaging said tool against said portion of said bottom; and
(c) moving said tool to generate said external force for causing destruction of said detaching member.

4. The improvement of claim 2 wherein said detaching member is made with polystyrene.
